(19) United States
(12) Patent Application Publication
(10) Pub. No.: US 2015/0283316 A1
(43) Pub. Date: Oct. 8, 2015

(54) MICRO-PILLAR ARRAY METHOD AND APPARATUS

(71) Applicant: LIGHTSTAT LLC, Southlake, TX (US)

(72) Inventors: Ayal Ram, Ramat Gan (IL); Oren Zinder, Ramat Gan (IL)

(21) Appl. No.: 14/443,475

(22) PCT Filed: Nov. 18, 2013
(86) PCT No.: PCT/US2013/070567

§ 371 Date: May 18, 2015

Related U.S. Application Data
(60) Provisional application No. 61/796,647, filed on Nov. 16, 2012.

(51) Int. Cl. A61M 1/36; B01D 21/00

(52) U.S. Cl. CPC A61M 1/36; B01D 21/0075

(57) ABSTRACT

A micro-pillar array assembly including an inlet for receiving a biological fluid including components of the biological fluid, an outlet, and a cavity formed between the inlet and the outlet, the cavity further comprising a plurality of micro-pillar arrays positioned to remove cellular components of the biological fluid to produce a resulting fluid. Another assembly, a method and a system are also disclosed.

### CROSS-REFERENCE TO RELATED APPLICATIONS

This application is a 371 national stage of PCT/US2013/070567 filed Nov. 18, 2013, which claims the benefit of U.S. Provisional Application No. 61/796,647 filed Nov. 16, 2012, and incorporated herein by reference in their entirety.

### BACKGROUND

Embodiments relate to a method and apparatus for performing non-centrifugal separation of whole blood components from plasma.

The investigation of blood fluid plasma and its components is a major medical diagnostic procedure carried out in clinical laboratories around the world. The investigation often involves the diagnosis of disease entities and the determination of possible prognosis. The investigation often uses large sophisticated instruments that require that a blood sample be taken from the patient, usually by venipuncture in combination with evacuated test tube, and subsequent centrifugation of the blood sample to obtain a required blood fluid (plasma or serum used in analysis. The fluid sample is then introduced into a clinical laboratory instrument for analysis after which the results are reported to a physician in the hospital, the clinic, or the doctor's office. On the basis of the test results, the physician will treat the patient or obtain an understanding of the severity of the disease. This process is also carried out for yearly check-ups or screening procedures for specific diseases.

The present clinical laboratory analysis is time consuming, relatively expensive, and requires venipuncture to obtain the sample. The results of the tests usually take one or more days to arrive at the physician's office and often require the patient to make a second visit to discuss the results and the subsequent therapy. In addition, the psychological fear of venipuncture makes the process tedious and may result in erroneous results due to the stress factor present during the venipuncture procedure. Finally, the venipuncture procedure and subsequent blood analysis requires trained operators such as phlebotomists, clinical laboratory professionals, and professionals trained in the use of sophisticated laboratory instruments, which drive up the cost and complexity of the analysis.

As a result, there is a need for a blood collection method and apparatus that reduces both the time and the cost of the blood collection and analysis.

### SUMMARY

In view of the foregoing considerations, Point of Care (POC) instruments are being developed, which carry out testing by the bedside or in the physician's office. An assembly, a method and a system are disclosed to provide for performing non-centrifugal separation of a resulting fluid from a whole fluid, such as, but not limited to, separation of plasma from whole blood. A micro-pillar array assembly comprises an inlet for receiving a biological fluid including components of the biological fluid, an outlet, and a cavity formed between the inlet and the outlet, the cavity further comprising a plurality of micro-pillar arrays positioned to remove cellular components of the biological fluid to produce a resulting fluid.

Another micro-pillar array assembly comprises an inlet for receiving whole blood including cellular components of the whole blood, an outlet, and a cavity formed between the inlet and the outlet, the cavity further comprising a plurality of micro-pillar arrays positioned to remove the cellular components of the whole blood to produce plasma.

The method comprises introducing a biological fluid into an inlet of a cavity structure comprising micro-pillar arrays wherein the biological fluid comprises at least one of whole blood, urine, saliva, or cerebral spinal fluid. The method also comprises selectively removing cellular components of the biological fluid with the micro-pillar arrays. The method also comprises capturing a resulting fluid at an outlet of the cavity structure in response to selectively removing the cellular components of the biological fluid with the micro-pillar arrays.

The system comprises a diagnostic apparatus configured to analyze plasma. The system also comprises a disposable extraction apparatus configured to remove the cellular components of the whole blood to produce the plasma, the extraction apparatus further comprising a plurality of micro-pillar arrays positioned to remove the cellular components of the whole blood to produce the blood plasma.

### BRIEF DESCRIPTION OF THE DRAWINGS

The accompanying drawings, which are incorporated into and form a part of the specification, illustrate one or more embodiments and, together with the description, serve to explain the principles of the invention. The drawings are only for the purpose of illustrating one or more embodiments and are not to be construed as limiting the invention. In the drawings:

FIG. 1A displays a view of an embodiment of a diagnostic system;

FIG. 1B displays a view of another embodiment of a diagnostic system;

FIG. 1C displays a view of another embodiment of a diagnostic system;

FIG. 2 displays a block diagram illustrating an embodiment of a diagnostic system;

FIG. 3A is a planar view of an arched implementation of a micro-pillar array assembly;

FIG. 3B is a perspective view of the micro-pillar array assembly of FIG. 3A;

FIG. 3C is an exploded view of a plurality of arrays 10 shown in FIG. 3B;

FIG. 3D is a perspective view of an arched implementation of a micro-pillar array assembly implemented with the array of triangular pillars shown in FIG. 3A;

FIG. 3E is an exploded view of the array of triangular pillars shown in FIG. 3D;

FIG. 4A is a planar view of a zig-zag implementation of a micro-pillar array assembly;

FIG. 4B is a perspective view of the zig-zag implementation of the micro-pillar array assembly shown in FIG. 4A;

FIG. 4C is an exploded view of the array of micro-pillars shown in FIG. 4B;

FIG. 4D is a perspective view of the arched implementation of micro-pillar array assembly implemented with arrays of triangular pillars shown in FIG. 4A;

FIG. 4E is an exploded view of the micro-pillar array assembly implemented with triangular pillars as shown in FIG. 4D;

FIG. 5A is a planar view of a W-type implementation of a micro-pillar array assembly;

FIG. 5B is a perspective view of the W-type micro-pillar array assembly of FIG. 5A;

FIG. 5C is an exploded view of the array of micro-pillars shown in FIG. 5B;

FIG. 5D is a perspective view of the micro-pillar array assembly of FIG. 6A;

FIG. 5E is an exploded view of FIG. 5D in which the micro-pillars arrays are implemented with triangular pillars;

FIG. 6A is a planar view of a slot-implementation of the micro-pillar array assembly;

FIG. 6B is a cross-sectional view of the micro-pillar array assembly of FIG. 6A along section A-A;

FIG. 6C is a perspective view of the micro-pillar array assembly shown in FIG. 6A;

FIG. 7A is a planar view of a zig-zag slot-implementation of the micro-pillar array assembly;

FIG. 7B is a cross-sectional view of the zig-zag slot-implementation of the micro-pillar array of FIG. 7A along section A-A;

FIG. 7C is a perspective view of the zig-zag slot-implementation of the micro pillar array assembly shown in FIG. 7A; and

FIG. 8 shows flowchart illustrating an embodiment of a method.

### DETAILED DESCRIPTION

Embodiments are described herein with reference to the attached figures, wherein like reference numerals are used throughout the figures to designate similar or equivalent elements. The figures are not drawn to scale and they are provided merely to illustrate aspects disclosed herein. Several disclosed aspects are described below with reference to non-limiting example applications for illustration. It should be understood that numerous specific details, relationships, and methods are set forth to provide a full understanding of the embodiments disclosed herein. One having ordinary skill in the relevant art, however, will readily recognize that the disclosed embodiments can be practiced without one or more of the specific details or with other methods. In other instances, well-known structures or operations are not shown in detail to avoid obscuring aspects disclosed herein. The embodiments are not limited by the illustrated ordering of acts or events, as some acts may occur in different orders and/or concurrently with other acts or events. Furthermore, not all illustrated acts or events are required to implement a methodology in accordance with the embodiments.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As an overview, a disposable diagnostic method and apparatus are presented. In an embodiment, a specific design is presented which includes a disposable component, a card, and a fluid introduction system. More specifically, a method and apparatus for capturing plasma from whole blood are presented. In an embodiment, a micro-pillar array assembly is presented. The micro-pillar array assembly includes a plurality of micro-pillar arrays organized to enable the extraction of specific components of whole blood. After whole blood is introduced into an inlet of the micro-pillar array assembly, cellular components of the blood are harvested as the blood flows through array structures positioned within the micro-pillar array assembly. In one embodiment, each array structure is positioned and sized to capture a specific cellular component of the whole blood. Non-limiting examples of cellular components include erythrocytes, leukocytes and platelets. Therefore, in a single assembly, during a single operation, whole blood components, namely cellular components, are harvested from the whole blood. Solid components may also be harvested. As a non-limiting example of solid components, membrane debris, which may be produced during an extraction of the whole blood from a living organism, is mixed with the whole blood.

Though the primary fluid or bodily fluid discussed herein is blood, primarily whole blood, embodiments may work with a variety of other fluids as well. As non-limiting examples with respect to biological fluids, such other fluids may include, but are not limited to, blood, urine cerebral spinal fluid (“CSF”), or saliva collected through various collection means such as venipuncture, urine container, or CSF from lumbar puncture.

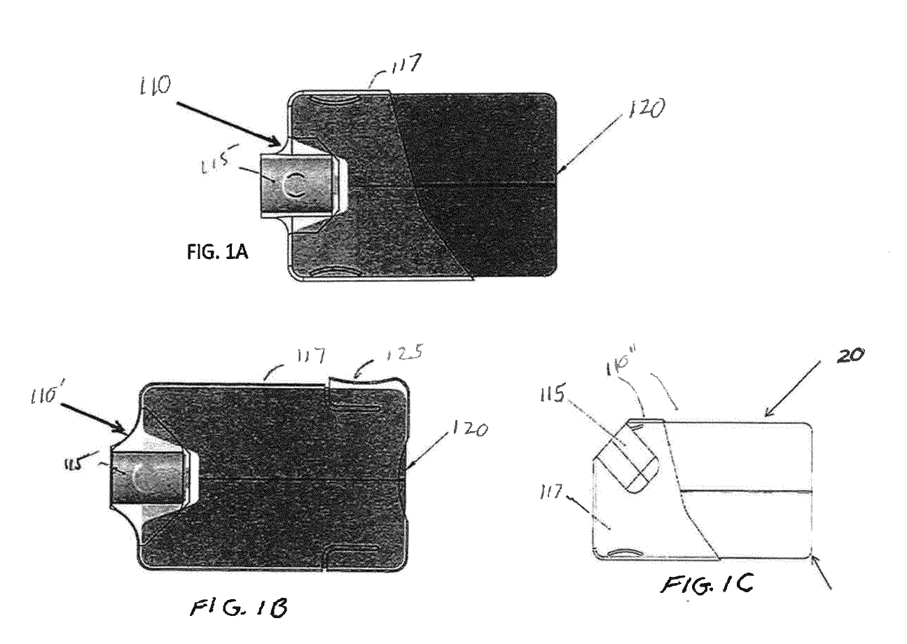

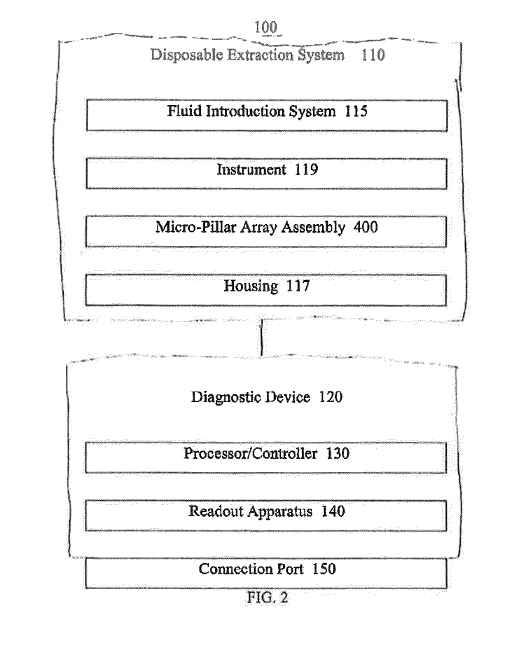

In an embodiment, a disposable diagnostic system is disclosed. FIGS. 1A, 1B, and 1C show embodiments of a variety of diagnostic system 100 designs. A disposable extraction system 110, 110′, 110″ or sample collection device may be provided. A diagnostic device 120 is provided. The diagnostic device 120 may be reusable. The diagnostic device 120 which may be a card, may comprise a controller 130 (illustrated in FIG. 2), such as, but not limited to, a microprocessor controller. Once a resulting fluid, specifically fluid which has passed through a micro-pillar-array assembly as disclosed herein, reaches an analysis location within the disposable extraction system 110, 110′, 110″, the diagnostic device 120 is able to perform an analysis on the resulting fluid. The diagnostic device 120 may further comprise a readout apparatus 140 (as illustrated in FIG. 2), such as, but not limited to, a readout screen or a connection port to connect the card 120 to another readout device.

The disposable extraction system 110, 110′, 110″ has a fluid introduction system 115 and a housing 117 to house the diagnostic device card 120. The fluid introduction system 115 may connect or attach to a fitting on the housing or may be integrated with the housing 117. The disposable extraction system 110, 110′, 110″ may comprise a plurality of configurations. As illustrated in FIG. 1A, the housing 117 may be configured to partially enclose the diagnostic device card 120. As illustrated in FIG. 1B, the housing 117 may be configured to fully enclose the diagnostic device 120 card wherein tabs 125 or detachment elements are provided to release the diagnostic device 120 when the disposable extraction system 110, and not the diagnostic device, is ready to be discarded. As illustrated in FIG. 1C with respect to FIGS. 1A and 1B, a location of the fluid introduction system 115 may be varied. The fluid introduction system 115 may be any fluid introduction system used to collect and introduce a variety of fluids into the disposable extraction system.

FIG. 2 shows an embodiment of a block diagram representing the disposable diagnostic system 100. The disposable extraction system 110 is disclosed with the fluid introduction system 115 and housing 117. An instrument 119 to exact, such as, but not limited to, by means of a prick or micro insertion into an epidermis layer of a living organism, the whole blood for the living organism may also be provided as part of the fluid introduction system. The whole blood may be provided to a micro-pillar array assembly 400, which is disclosed in further detail herein. The micro-pillar array assembly may be a part of the disposable extraction system 110.

A diagnostic device 120 is disclosed. In an embodiment, the diagnostic device is a portable diagnostic device. “Portable” is provided to suggest that the device may be handheld and easily transportable by a single individual, unaided. The diagnostic device 120 may be a card. A processing capability, such as, but not limited to, a controller or microprocessor controller 130 is provided. A readout apparatus 140 is shown. The readout apparatus 140 may be a screen used to display the analyzed results. The term “screen” and “display” are not meant to be limiting as information from analysis performed by the diagnostic device 120 may be communicated visually, audibly, or tactilely. Thus, the screen may be simply considered a communication apparatus which can display resulting analysis in any form explained herein. As such the use of screen and display encompasses devices which may operate based on at least one of these forms of communication. Also a connection port 150, or external port, such as, but not limited to, a universal serial bus (“USB”) port (either a male or female receptacle) may be provided to connect the diagnostic device 120 to another display apparatus (not shown), such as, but not limited to, a computer having a monitor, a computing tablet, a smart phone, etc. The readout apparatus 140 may provide for visual or audible display.

Thus, the disposal extraction system in combination with the diagnostic device provides for a micro-analytical device, which processes very small blood samples and provides sufficient clear and pure plasma for effective and accurate analyses of the components found in the fluid of the whole blood.

As is disclosed in further detail below with respect to the teachings herein, in an embodiment, blood cells are separated from plasma without the need for an external filtering device. The separation is based on the mechanical blocking of cells as the cells are processed through a multi-tier micro-pillar assembly. The micro-pillar assembly comprises arrays of cylindrical micro-pillars organized into a multi-tier assembly. In an embodiment, the arrays may comprise a plurality of micro-pillars arranged in a straight line. However, it should be appreciated that off-line arrangements, where the micro-pillars are nearly in a straight line or not in a straight line, such as slanted or sloped, are also contemplated by the teachings disclosed herein.

In another embodiment, an array may be considered as a capture point within a cavity of a micro-pillar assembly, where cellular components of a liquid such as blood are blocked from further transport through the micro-pillar array assembly and may be captured for further analysis. Each array creates a tier of the multi-tier micro-pillar array assembly. As a result, the cellular components of whole blood may be blocked and captured at the various tiers of the multi-tier micro-pillar assembly. Solid components may also be captured.

Each tier of the multi-tier micro-pillar array assembly may include pillars which are spaced closer together as you traverse the tiers of the micro-pillar array assembly from an inlet of the multi-tier micro-pillar assembly to an outlet of the multi-tier micro-pillar assembly. Specifically, in a single array, the spacing between the pillars is established to capture cells of a specific size. As a non-limiting example, the pillars in the first tier may be spaced at about 8 microns apart and the pillars at the next tier may be spaced at about 5 microns apart, etc. As such, cells that are smaller than about 8 microns will be able to pass through the first tier, but would be stopped and collected at the second tier if the cells are greater than or equal to about 5 microns.

Whole blood is introduced into a micro-pillar assembly. As the whole blood passes through the multi-tier micro-pillar array assembly, cells of varying sizes are blocked as a result of the size of the cell relative to the size of the spacing between the pillars in the array. The blocked blood cells are then analyzed. In an embodiment, whole blood is introduced into the inlet of a multi-tier micro-pillar array assembly and plasma is collected at the outlet of the multi-tier micro-pillar array assembly.

In an embodiment of the micro-pillar array assembly the closest array to the inlet of the micro-pillar array which engages the whole blood includes pillars that are about 30 microns apart (i.e., between pillars). The distance between pillars is then gradually decreased, for example, subsequent arrays would have pillars with a distance between pillars of about 25 microns, about 20 microns, about 15 microns, about 10 microns, and about 5 microns, respectively. In one embodiment, this reduction in the distance would decrease to about 2 microns between pillars.

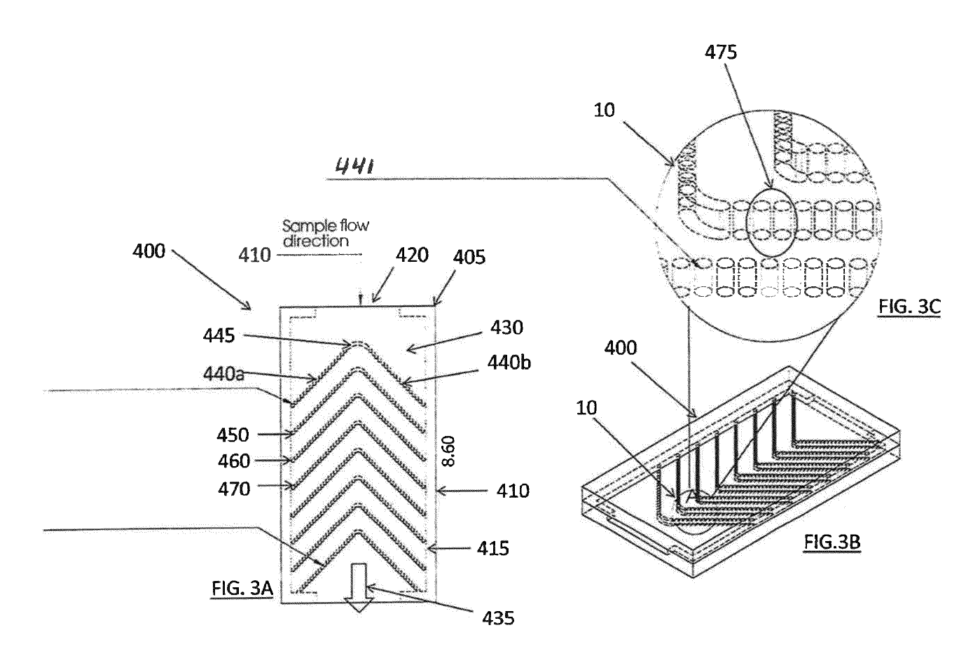

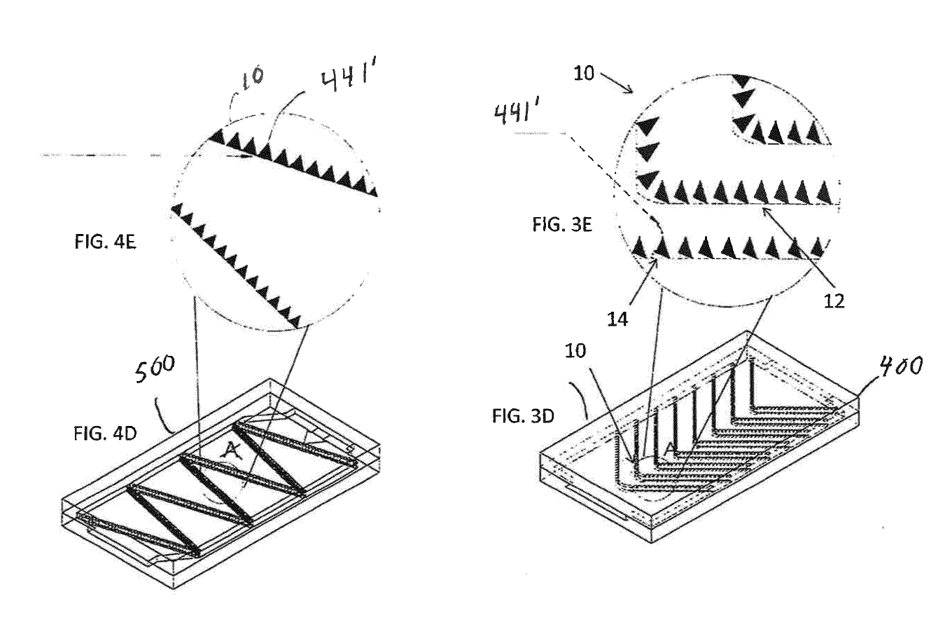

FIG. 3A shows a planar view of an arched micro-pillar array assembly implemented in accordance with the teachings of the present invention. In FIG. 3A a micro-pillar array assembly 400 is shown. The micro-pillar array assembly 400 includes a micro-pillar array casing 405 which may include an outer wall 410 and an inner wall 415. The inner wall 415 creates a cavity 430 within the micro-pillar array assembly 400. An inlet 420 to the cavity 430 and an outlet 435 to the cavity 430 are shown. An array of pillars 440a and 440b are angled and separated from each other by a ridge 445. The combination of the matching array of pillars 440a, 440b, and the ridge 445 are referred to as an arched array, for the purposes of this disclosure. In one embodiment, the arched arrays are replicated in parallel as shown by 450, 460, and 470 within the cavity 430 of the micro-pillar array assembly 400.

During operation, whole blood is introduced into the cavity 430 through the inlet 420 in the direction of flow depicted by 410. The whole blood flows across the ridge 445 and on to the arrays 440a and 440b. In one embodiment, two activities will occur with the whole blood. Cells that cannot pass through the array of pillars 440a and 440b, due to their size relative to the spacing of the pillars, will be blocked, gently roll down the sides of the array of pillars 440a and 440b and be captured. Plasma and cells that can pass through the pillars in the array of pillars 440a and 440b will impact the next array of pillars 450, where the same process will occur. Some cells will be captured, while the other cells and plasma will continue to traverse through the cavity 430 micro-pillar array assembly 400.

As a result of the foregoing process, whole blood is input into the inlet 420 and transformed into plasma at the outlet. In one embodiment, each column of cylinders 440 has smaller spacing between the cylinders. As such, as the whole blood traverses the column of cylinders various sized blood cells are captured starting with the larger blood cells down to the smallest size blood cells, before plasma is produced.

In one embodiment of the multi-tier micro-pillar array assembly, arrays are organized to extract plasma from whole blood. The arrays are arranged as parallel sets of arrays in Which each subsequent array, as you move from the inlet to the outlet of the micro-pillar array assembly, includes arrays with smaller distances between the pillars. In one embodiment, the arrays include pillars with a pillar-to-pillar distance (i.e., distance between pillars) of approximately 10 microns which will effectively block passage of the largest cells. The middle-tier array includes pillars with a pillar-to-pillar distance of approximately 5 microns, which will block passage of all, but the red blood cells. A final array includes pillars with a pillar-to-pillar distance of approximately 2 microns between pillars, which will effectively block passage of red blood cells, the smallest of all blood cells. In one embodiment, the pillar height is approximately 40 microns and is confined by an upper plate to contain the whole blood within a cavity of the micro-pillar array assembly. It should be appreciated that although blood is used as an example, use of the micro-pillar array assembly to remove cellular components, and/or even solid components, of any liquid is contemplated and within the scope of the embodiments disclosed herein.

In one embodiment, an arched arrangement of arrays is disclosed. With the arched arrangement of arrays, once the cells are captured by the pillars, the captured cells gently roll down the sides of the array and rest at the bottom of the array within the cavity of the micro-pillar array assembly. This will result in the separation of the captured cells into two groups, one on each side of array cavity, and thus the cells will be divided into six separate pools (i.e., when a first, second, and third tier scheme is implemented). The cells will thus be in small pools and will have a gross separation according to size. In one implementation, an exit port will be implemented through which the cells will be able to roll out of the cavity of the micro-pillar array assembly, or to be actively extracted. This will allow for subsequent analysis of the different blood cells, which have been separated grossly by size. The separated plasma will be produced at the outlet of the micro-pillar array assembly and enter a chamber with a finite volume (i.e., the size of which is determined by the amount of plasma needed for any specific test). In one embodiment, any excess plasma will be diverted into a waste micro-channel and deposited in a closed waste chamber.

FIG. 3B is a perspective view of the micro-pillar array 400 of FIG. 3A. In FIG. 3B an exploded view 10 of an array of pillars is displayed. FIG. 3C is the exploded view 10 of the array of pillars shown in FIG. 3B. In one embodiment, it should be noted that the space between pillars 441 denoted by 475 of FIG. 2C varies in size. In other words, the distance between the pillars 475 varies. In one embodiment, the space between the pillars shown as 475 of FIG. 3C is sized to capture the different components, but namely cellular components, of whole blood. In one embodiment the space between the pillars 475 of FIG. 3C is uniform in any single array, but decreases between arrays. In another embodiment, the space between the pillars 475 of FIG. 3C is non-uniform between arrays. In yet another embodiment, the space between the pillars 475 of FIG. 3C vary from 0.008 in a first array (i.e., array closes to the inlet 420) to 0.002 in a last array (i.e., array closest to the outlet 435) in a micro-pillar array 400 of FIG. 3A.

FIG. 3D is a perspective view of an arched implementation of a triangular micro-pillar array assembly implemented with triangular pillars. FIG. 3F is an exploded view 10 of the triangular pillars shown in FIG. 3D. In one embodiment, the triangular pillars 441′ form a triangular shape when a cross-section of the triangular pillars 441′ is taken. In accordance with the teachings of the present invention, the cross-sectional dimension of the triangular pillars 441′ may be implemented with a variety of different types of triangles including, but not limited to, scalene triangles, equilateral triangles, obtuse triangles, right triangles, etc. In one embodiment, the micro-pillar array assembly implemented with triangular pillars 441′ may include a base of the triangle (i.e., in the cross-sectional view) where the base of the triangle sits flush with a reference line 12. In a second embodiment, a base of the triangle (i.e., in the cross-sectional view) may be oriented relative to the reference line 12 to form an angle shown as 14. It should be appreciated that in accordance with the teachings of the present invention a cross section of a micro-pillar array may be oriented in a variety of positions relative to a reference line. Further, each micro-pillar 441 may be oriented in the same way or a variety of micro-pillar sizes and orientations may be implemented in accordance with the teachings disclosed herein.

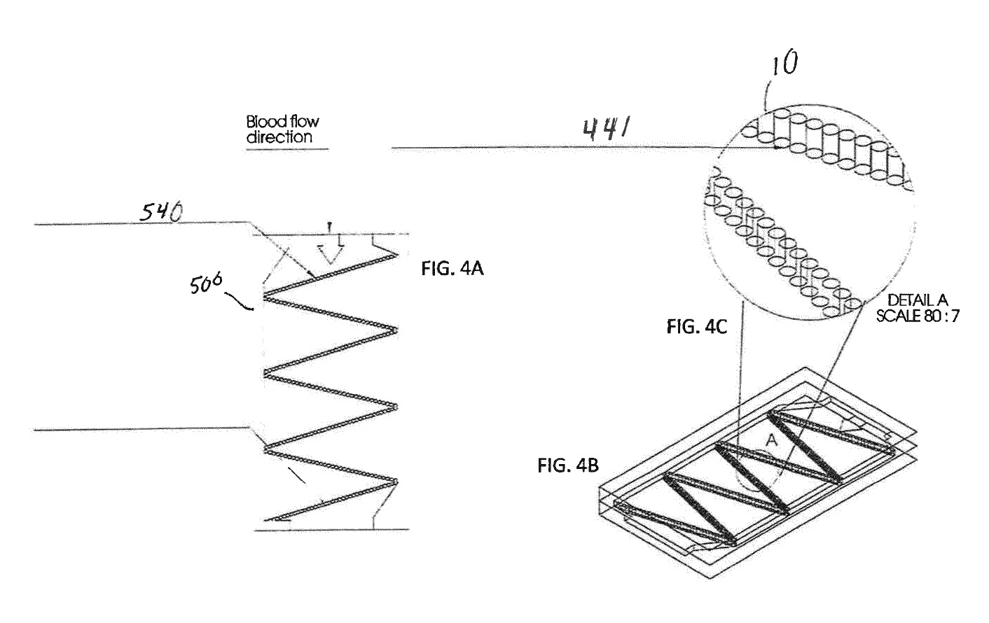

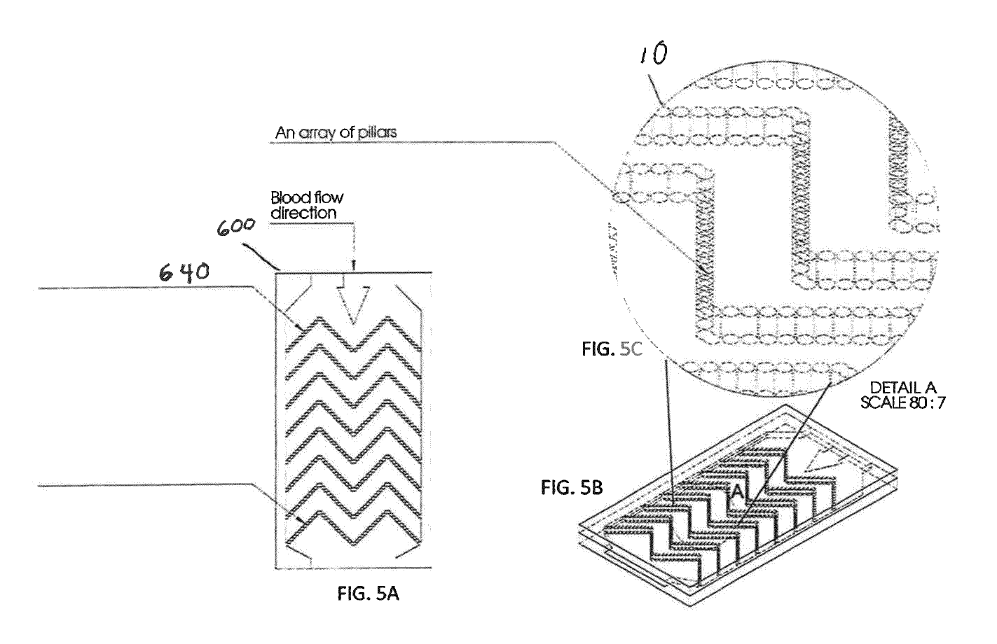

FIG. 4A is a planar view of a micro-pillar array assembly, where the arrays are positioned in a zig-zag pattern. FIG. 4B displays a perspective view of the micro-pillar array 500 of FIG. 4A, where the arrays are positioned in a zig-zag pattern. FIG. 4C displays an exploded view of the micro-pillar array assembly shown in FIG. 4B with cylindrical pillars 441. In place of the cylindrical pillars 441, FIG. 4D shows a perspective view of the zig-zag implementation of a triangular micro-pillar array implemented with triangular pillars 441′. FIG. 4E is an exploded view 10 of the triangular pillars 441′ shown in FIG. 4D.

In accordance with the teachings herein, the cross-sectional dimension of the triangular pillars 441′ may be implemented with a variety of different types of triangles including, but not limited to, scalene triangles, equilateral triangles, right triangles, right triangles, etc. In one embodiment, the triangular micro-pillars 441′ may include a base of the triangle that sits flush with a reference line 12 or a base of the triangle may be oriented relative to the reference line 12 to form an angle. It should he appreciated that in accordance with the teachings herein a cross section of a micro-pillar array may be oriented in a variety of positions relative to a reference line. Further, each micro-pillar array may be oriented in the same way or a variety of micro-pillar sizes and orientations may be implemented in accordance with the teachings disclosed herein. Further, it should be appreciated that non-circular and non-triangular cross-sectional micro-pillar shapes may be implemented in accordance with the teachings herein, such as, but not limited to, oblong shapes, square shapes, half-circle shapes, etc.

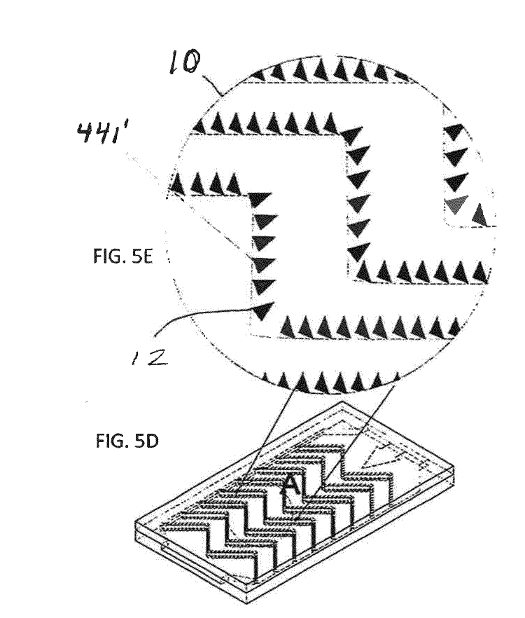

FIG. 5A is a planar view of a W-type implementation of a micro-pillar array 640. FIG. 5B is a perspective view of the W-type implementation of the micro-pillar arrays 640 of the micro-pillar array assembly 600 of FIG. 5A with cylindrical pillars 441. FIG. 5C is an exploded view 10 the array of pillars shown in FIG. 5B. FIG. 5D is a perspective view of a W-type micro-pillar array assembly implemented with triangular pillars 441′. FIG. 5E is an exploded view of the triangular pillars 441′ shown in FIG. 5D.

In accordance with the teachings herein, the cross-sectional dimension of the triangular pillars 441′ may be implemented with a variety of different types of triangles including, but not limited to, scalene triangles, equilateral triangles, right triangles, right triangles, etc. As shown in FIG. 5E, in one embodiment, a cross-section of the triangular micro-pillar 441′ may include a base of the triangle that sits flush with a reference line 12 or a base of the triangle may be oriented relative to the reference line to form an angle. It should be appreciated that in accordance with the teachings herein, a cross section of a micro-pillar array may be oriented in a variety of positions relative to the reference line 12. Further, each micro-pillar 441′ may be oriented in the same way or a variety of micro-pillar sizes and orientations may be implemented in accordance with the teachings disclosed herein. Further, each micro-pillar array 441′ may be oriented in the same way or a variety of micro-pillar sizes and orientations may be implemented in accordance with the teachings disclosed herein. Further, it should be appreciated that non-circular and non-triangular cross-sectional micro-pillar shapes may be implemented in accordance with the teachings of the present invention, such as oblong shapes, square shapes, half-circle shapes, etc.

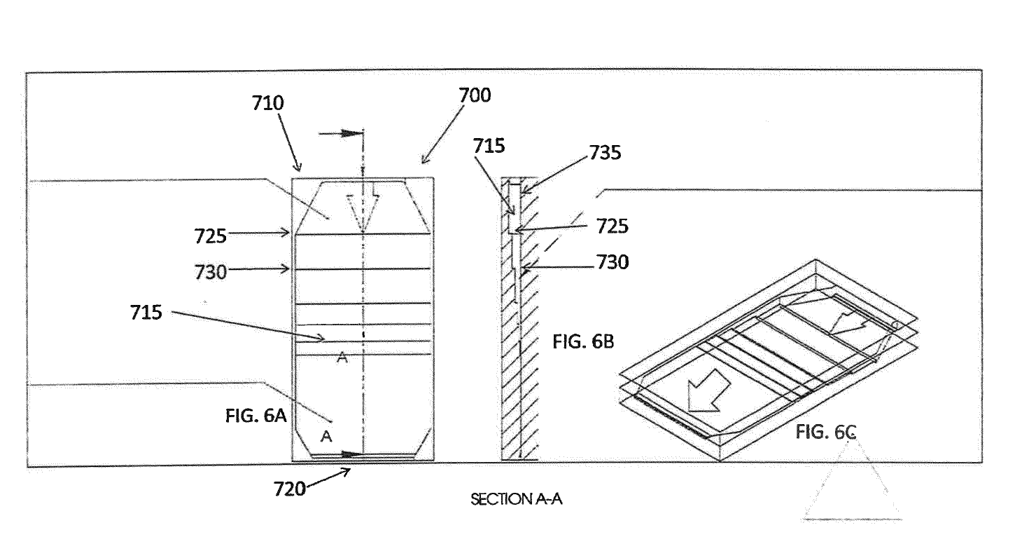

FIG. 6A is a planar view of a slot-implementation of the micro-pillar array assembly. FIG. 6B shows a cross section of the slot-implementation of the micro-pillar array assembly 700 shown in FIG. 6A along sectional line A-A shown in FIG. 6A. In FIG. 6A a liquid such as blood flows from an inlet 710 through a slotted-cavity 715 to an outlet 720. As previously mentioned, in one embodiment of the present invention an array may be considered as a capture point within a cavity of a micro-pillar assembly where cellular components of a liquid such as blood are blocked and captured for further analysis. In one embodiment, the cavity 715 forms a variation of a v-shape when viewed at cross section A-A. As such, the opening at the inlet 710 is greater than the opening at the outlet 720. In other words, the cavity 715 decreases in cross-sectional size from inlet 710 to outlet 720. As liquid traverses through the cavity 715, various ridges, such as a first ridge 725 and a second ridge 730 are defined. In the slot-implementation 700 of the micro-pillar array assembly, the first ridge 725 is considered a first array and second ridge 730 is considered a second array.

In FIG. 6B a cross-sectional view along line A-A of FIG. 6A is shown. A cavity 715 is defined in the cross-sectional view. In the cavity 715, a first ridge 725 and the second ridge 730 define a cavity width of different sizes relative to an oppositely disposed wall of the cavity 735. As a non-limiting example, in one embodiment, the cavity width as defined by the first ridge 725 and the oppositely disposed wall of the cavity 735 about 8 microns. The cavity width defined by the second ridge 730 and the oppositely disposed wall of the cavity 735 is a value less than about 8 microns such as 5 microns. In one embodiment of the slot-implemented micro-pillar array assembly of FIG. 6A the cavity widths decrease in size to capture various cellular components of whole blood such as the red blood cells, the white blood cells, and platelets. FIG. 6C is a perspective view of the slot-implementation 700 of the micro-pillar array assembly.

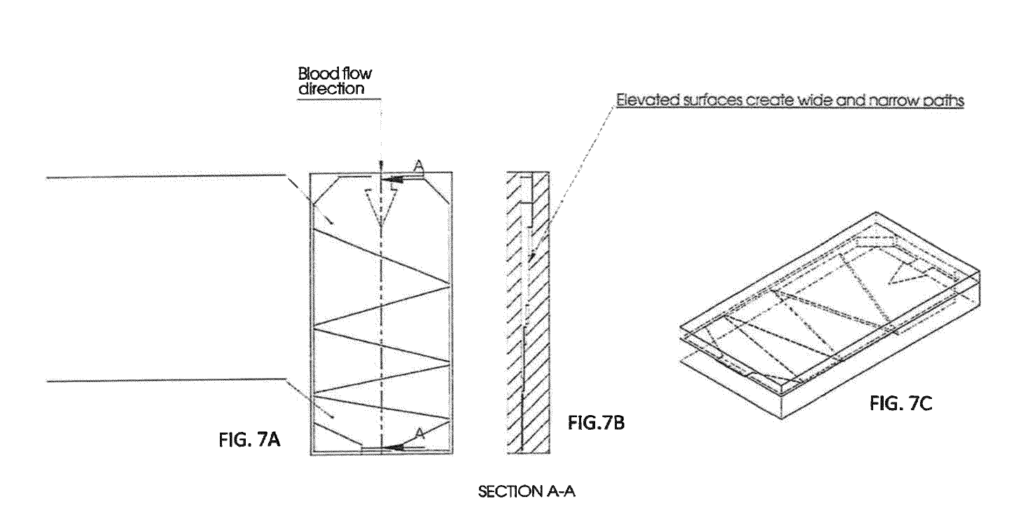

FIG. 7A is a planar view of a slot-implementation of the micro-pillar array assembly with a zig-zag formation. FIG. 7B is a slot-implementation of the micro-pillar array assembly with the zig-zag formation shown in FIG. 7A along sectional line A-A. FIG. 7C is a perspective view of the slot-implementation of the micro-pillar array assembly shown in FIG. 7A.

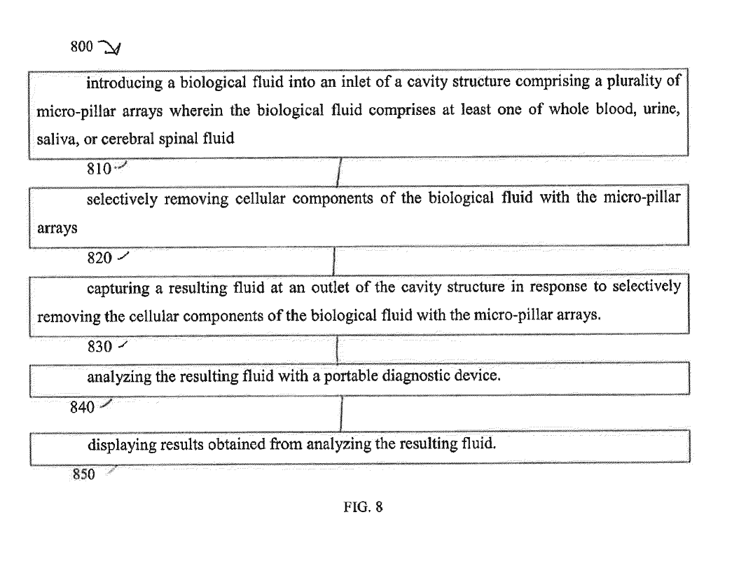

FIG. 8 shows flowchart illustrating an embodiment of a method. The method 800 comprises introducing a biological fluid into an inlet of a cavity structure comprising micro-pillar arrays wherein the biological fluid comprises at least one of whole blood, urine, saliva, or cerebral spinal fluid, at 810. The method also comprises selectively removing cellular components of the biological fluid with the micro-pillar arrays, at 820. The method further comprises capturing a resulting fluid at an outlet of the cavity structure in response to selectively removing the cellular components of the biological fluid with the micro-pillar arrays, at 830.

With respect to blood or whole blood, the resulting fluid is plasma. Furthermore, as used herein, “removing cellular components” and “remove cellular components” do not mean that all cellular components are removed since some cellular components may remain. Therefore, these terms should not be considered absolutes.

In an embodiment, selectively removing the cellular components, at 820, may further comprise removing the cellular components with the micro-pillar arrays having a configuration selected from the group consisting of an arched configuration, a zig-zag configuration, a w-type configuration, slanted configuration, and a slotted configuration. In an embodiment, selectively removing the cellular components, at 820, further comprises removing the cellular components with the micro-pillar arrays comprising pillars having a cross section of at least one of circular, non-circular, or triangular.

The method 800 may further comprise analyzing the resulting fluid with a portable diagnostic device, 840. The method 800 may further comprise displaying results from analyzing the resulting fluid, at 850. As disclosed herein, displaying results may be performed in one of a plurality of ways. Though the steps illustrated above and provided in a particular sequence, this sequence is not meant to be limiting as those skilled in the art will recognize that these steps may be performed in any particular order.

Thus, as taught herein in one embodiment, a method and apparatus are disclosed which enable the rapid and complete separation of plasma (i.e., blood fluid) from whole blood by separating and positioning the components of the whole blood so that they can be harvested for further analysis. As a result, the whole blood cellular components including the plasma may be used for clinical laboratory analysis. As a non-limiting example, the blood cells separated from the blood fluid (plasma) can be harvested and used to investigate blood cell disorders. In one embodiment, the components of whole blood are the cellular components. However, it should be appreciated that the molecular components of whole blood are also considered and contemplated. Another component may also be solid components.

In another embodiment, the teachings herein provide for blood cells being segregated in a manner that enable them to be harvested for further analysis leading to determination of potential blood cell diseases. In another embodiment, a single device is disclosed which enables the seamless separation of the plasma and the analysis of whole blood components, namely cellular components or the plasma, within a single instrument. In an embodiment, separating the biological fluid is accomplished, without the need for increased gravity, such as through the use of a centrifuge.

While various disclosed embodiments have been described above, it should be understood that they have been presented by way of example only, and not limitation. Numerous changes to the subject matter disclosed herein can be made in accordance with the embodiments disclosed herein without departing from the spirit or scope of the embodiments. In addition, while a particular feature may have been disclosed with respect to only one of several implementations, such feature may be combined with one or more other features of the other implementations as may be desired and advantageous for any given or particular application. Therefore, the breadth and scope of the subject matter provided herein should not be limited by any of the above explicitly described embodiments. Rather, the scope of the embodiments should be defined in accordance with the following claims and their equivalents.

The terminology used herein is for the purpose of describing particular embodiments only and is not intended to be limiting. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.” Moreover, unless specifically stated, any use of the terms first, second, etc., does not denote any order or importance, but rather the terms first, second, etc., are used to distinguish one element from another.

Unless otherwise defined, all terms including technical and scientific terms) used herein have the same meaning as commonly understood by one of ordinary skill in the art to which embodiments of the invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Thus, while embodiments have been described with reference to various embodiments, it will be understood by those skilled in the art that various changes, omissions and/or additions may be made and equivalents may be substituted for elements thereof without departing from the spirit and scope of the embodiments. In addition, many modifications may be made to adapt a particular situation or material to the teachings of the embodiments without departing from the scope thereof. Therefore, it is intended that the embodiments not be limited to the particular embodiment disclosed as the best mode contemplated, but that all embodiments falling within the scope of the appended claims are considered.

What is claimed is:

1. A micro-pillar array assembly, comprising:
an inlet for receiving a biological fluid including components of the biological fluid;
an outlet; and
a cavity formed between the inlet and the outlet, the cavity further comprising a plurality of micro-pillar arrays positioned to remove cellular components of the biological fluid to produce a resulting fluid.

2. The micro-pillar array assembly according to claim 1, wherein the biological fluid comprises Hood, urine, cerebral spinal fluid, or saliva.

3. The micro-pillar array assembly according to claim 1, wherein respective micro-pillar arrays of the plurality of micro-pillar arrays are arranged in a sequence between the inlet and the outlet.

4. The micro-pillar array assembly according to claim 1, wherein at least one of the plurality of micro-pillar arrays is implemented with a configuration selected from a group consisting of an arched configuration, a zig-zag configuration, a w-type configuration, slanted configuration, and a slotted configuration.

5. The micro-pillar array assembly according to claim 1, wherein each one of the plurality of micro-pillar arrays further comprises pillars with varying spacing.

6. The micro-pillar array assembly according to claim 1, wherein each of the plurality of micro-pillar arrays further comprises pillars and wherein a cross section of the pillars is at least one of circular, non-circular, or triangular.

7. The micro-pillar array assembly according to claim 1, wherein the plurality of micro-pillar arrays comprises ridges that define varying widths between a first wall of the cavity and an oppositely disposed wall of the cavity.

8. The micro-pillar array assembly according to claim 9, wherein the ridges form a zig-zag pattern.

9. The micro-pillar array assembly according to claim 1, wherein the cavity further comprises a respective ridge configured to separate a first part and a second part of an array of pillars of the plurality of array of pillars.

10. A micro-pillar array assembly, comprising:
an inlet for receiving whole blood including components of the whole blood;
an outlet; and
a cavity formed between the inlet and the outlet, the cavity further comprising a plurality of micro-pillar arrays positioned to remove cellular components of the whole blood to produce plasma.

11. The micro-pillar array assembly according to claim 1, wherein at least one of the plurality of micro-pillar arrays positioned to remove the components of the whole blood is implemented with a configuration selected from a group consisting of an arched configuration, a zig-zag configuration, a w-type configuration, slanted configuration, and a slotted configuration.

12. The micro-pillar array assembly according to claim 1, wherein the micro-pillar arrays are further positioned to remove solid components from the whole blood.

13. The micro-pillar array assembly according to claim 1, wherein each of the plurality of micro-pillar arrays positioned to remove the cellular components of the whole blood further comprises pillars with varying spacing.

14. The micro-pillar array assembly according to claim 1, wherein the plasma is collected at the outlet after the whole blood traverses the plurality of micro-pillar arrays positioned to remove the components of the whole blood.

15. The micro-pillar array assembly according to claim 1, wherein each of the plurality of micro-pillar arrays positioned to remove the components of the whole blood further comprises pillars and wherein a cross section of the pillars is at least one of circular, non-circular, or triangular.

16. The micro-pillar array assembly according to claim 1, wherein the cellular components of the whole blood is at least one of molecular components, red blood cells, white blood cells, and platelets.

17. A method, comprising:
introducing a biological fluid into an inlet of a cavity structure comprising a plurality of micro-pillar arrays wherein the biological fluid comprises at least one of whole blood, urine, saliva, or cerebral spinal fluid;
selectively removing cellular components of the biological fluid with the micro-pillar arrays; and
capturing a resulting fluid at an outlet of the cavity structure in response to selectively removing the cellular components of the biological fluid with the micro-pillar arrays.

18. The method according to claim 17, wherein selectively removing the cellular components further comprises removing the cellular components with the micro-pillar arrays arranged in a sequence between the inlet and the outlet.

19. The method according to claim 17, wherein selectively removing the cellular components further comprises removing the cellular components with at least one of the plurality of micro-pillar arrays having at configuration selected from a group consisting of an arched configuration, a zig-zag configuration, a w-type configuration, slanted configuration, and a slotted configuration.

20. The method according to claim 17, wherein selectively removing the cellular components further comprises removing the cellular components with the micro-pillar arrays comprising pillars having a cross section of at least one of circular, non-circular, or triangular.

21. The method according to claim 17, further comprising analyzing the resulting fluid with a portable diagnostic device.

22. The method according to claim 21, further comprising displaying results obtained from analyzing the resulting fluid.

23. A system, comprising:
a diagnostic apparatus configured to analyze plasma;
a disposable extraction apparatus configured to remove cellular components of whole blood to produce the plasma, the extraction apparatus further comprising a plurality of micro-pillar arrays positioned to remove the cellular components of the whole blood to produce the blood plasma.

24. The system according to claim 23, wherein the disposable extraction apparatus further comprising a housing to hold the diagnostic card.

25. The system according to claim 23, wherein the disposable extraction apparatus further comprises a fluid introduction system configured to provide the whole blood to the micro-pillar arrays.

26. The system according to claim 23, wherein the diagnostic apparatus further comprises a communication apparatus to display blood plasma analysis.

27. The system according to claim 23, wherein the diagnostic apparatus further comprises an attachment location configured to connect the diagnostic card to a display device.

28. The system according to claim 23, wherein the disposable extraction apparatus further comprises an instrument to extract the whole blood for a living organism.

29. The system according to claim 23, wherein the disposable extraction apparatus further comprises a solid component extraction device configured to remove solid components from the whole blood.
